(19) United States
(12) Patent Application Publication
(10) Pub. No.: US 2018/0029079 A1
(43) Pub. Date: Feb. 1, 2018

(54) APPARATUS AND METHOD FOR PAIN RELIEF USING ULTRASOUND ENERGIZED POLYMERS

(71) Applicant: Eliaz Babaev, Minnetonka, MN (US)

(72) Inventor: Eliaz Babaev, Minnetonka, MN (US)

(21) Appl. No.: 15/460,285

(22) Filed: Mar. 16, 2017

Related U.S. Application Data
(63) Continuation of application No. 15/059,409, filed on Mar. 3, 2016, now abandoned.
Application No. 15/059,409 is a continuation of application No. 11/409,818, filed on Apr. 24, 2006, now abandoned.

(51) Int. Cl. B06B 3/04; B65B 5/04; B06B 1/02; B65B 63/00; A61K 41/00

(52) U.S. Cl. CPC B06B 3/04; B65B 63/00; B65B 5/045; A61K 41/0014; B06B 1/0223

(57) ABSTRACT

Method and device to create energized polymers that can be used for pain relief, comprised of an ultrasound system that ultrasonically energize polymers that can then be used to provide an analgesic effect. Ultrasound waves are delivered to a polymer through direct contact, through a coupling medium, or without contact in order to energize the polymer. Other energies such as such as UV, microwave, laser, electricity, RF, sun, light, magnetic/electromagnetic, et can also be used to energize the polymer. The energized polymer can be immediately placed on a user to provide an analgesic effect, or the energized polymer can be placed storage material and removed at a later time to be placed on a user to provide an analgesic effect.

### BACKGROUND OF THE INVENTION

### Field of the Invention

The present invention relates to pain relief. In particular, the present invention relates to apparatus and methods for pain relief using polymers energized by exposure to ultrasonic waves, and said polymers are capable of storing the energy imparted to them from ultrasound exposure.

### Description of the Related Art

Treating persistent lingering pain, often but not exclusively associated with arthritis, muscles soreness, headache, etc, with various forms of energy is well known to the art. Most often the energy chosen is a variant of thermal energy, which in particular is heat or cold applied via a portable pad or pack. Applying thermal energy to a portable pack or pad is generally accomplished by means of a chemical reaction or energy transfer by placing the pad or pack in hot environment, such as boiling water or a microwave oven, or a cold environment, such as a fridge or freezer. Transferring thermal energy to a portable pad or pack often results in the pad or pack becoming overheated or overcooled. When placed on the user, an overheated pad or pack can cause the user discomfort or burn the user's skin. Similarly, an overcooled pad or pack when placed on the user's body can cause the user discomfort or freeze burn the user's skin.

Supplying thermal energy to a portable pad or pack can also be accomplished by placing two or more chemicals that are temporarily separated within the pack or pad—these chemicals can be combined to create an endothermic or exothermic chemical reaction. When the user is in need of pain relief, the user activates the pad or pack by removing the barrier separating the reactive chemicals. Though effective at producing thermal energy, the use of chemicals in portable packs or pads is hazardous in that the chemicals employed can injure the user's skin if the chemicals were to leak out of the pad or pack.

Imparting thermal energy to a location of persistent lingering pain is also accomplished by applying chemicals and creams to the affected area and allowing them to evaporate. Though not effective at generating heat, the evaporation of chemicals applied to the skin can generate a local cooling at the location of the user's body experiencing persistent lingering pain. The use of creams and chemicals is disadvantaged by the fact that such creams and chemicals are often messy to apply and can cause severe irritation if they come in contact with the user's eyes or mucosal membranes.

Generating and applying therapeutic energy to a location of the body experiencing persistent lingering pain is also accomplished by electrical stimulation. Transcutaneous

Electrical Nerve Stimulation (TENS) is an example of this methodology. TENS, and other similar methods, treat pain by using electrodes to induce a current across the user's skin that transverses the site of persistent lingering pain. Portable versions of TENS, and similar devices, have been created and marketed. Requiring batteries or an external power source and often being bulking, TENS devices are not truly portable. Furthermore, the device is worthless if the user of the device is without batteries or an electrical outlet.

The limitations of the current energy based treatments of persistent lingering pain create a need for a portable device that is not bulky, that does not require the user to supply an external energy source or battery, that does not derive thermal energy from chemicals that irritate, injury, or burn the user's skin, and that cannot be overheated or overcooled as to avoid injuring the user.

### SUMMARY OF THE INVENTION

The present invention is directed towards apparatus and methods for pain relief by using polymers energized by exposure to ultrasound, and said polymers are capable of storing the energy imparted to them from ultrasound exposure. Apparatus and methods in accordance with the present invention may meet the above-mentioned needs and also provide additional advantages and improvements that will be recognized by those skilled in the art upon review of the present disclosure.

The present invention comprises an ultrasonic generator, an ultrasonic transducer, an ultrasound horn, and an ultrasound tip. Exposing a polymer to ultrasonic waves energizes the polymer and that polymer can then be used to provide pain relief.

Ultrasonic waves are delivered to a polymer in order to energize that polymer. Ultrasonic waves are delivered by directly contacting the polymer with the ultrasound tip, by contacting the polymer through a coupling medium, or without contacting the polymer. The energized polymer is applied to a user to provide an analgesic effect either immediately after being energized or the energized polymer can be stored for use at a future time.

The invention is related to apparatus and methods for pain relief that uses polymers energized by exposure to ultrasonic waves.

One aspect of this invention may be to provide a method and device for quick pain relief

Another aspect of this invention may be to provide a method and device for more effective pain relief.

Another aspect of the invention may be to provide a method and device for more efficient pain relief.

Another aspect of the invention may be to provide a method and device for safer pain relief.

Another aspect of the invention may be to provide a method and device for pain relief that does not use chemicals or drugs.

Another aspect of this invention may be to provide a method and device for pain relief that is easy to use.

Another aspect of the invention may be to provide a method and device for pain relief that can be used at home by an individual.

Another aspect of the invention may be to provide a portable means for pain relief.

These and other aspects of the invention will become more apparent from the written descriptions and figures below.

### BRIEF DESCRIPTION OF THE DRAWINGS

The present Invention will be shown and described with reference to the drawings of preferred embodiments and clearly understood in details.

FIG. 1 is a perspective view for an ultrasound apparatus capable of energizing polymers according to the present invention.

FIG. 2 is a cross-sectional view of the ultrasound apparatus.

FIG. 3 are front-views of ultrasound tips that can be used with the ultrasound apparatus.

FIG. 4 is a perspective view of an ultrasound apparatus capable of energizing polymers through direct contact with a polymer.

FIG. 5 is a detailed view of an ultrasound apparatus that can energize polymers through direct contact with a polymer.

FIG. 6 is a perspective schematic view of a production line with an ultrasound apparatus capable of energizing polymers through direct contact.

FIG. 7 is a perspective schematic view of a production line with an ultrasound apparatus capable of energizing polymers through a coupling medium.

FIG. 8 s a perspective schematic view of an example production line with an ultrasound apparatus capable of energizing polymers and with a separate device to seal polymers in storage.

FIG. 9 is a perspective view of a production line with an ultrasound apparatus capable of both energizing polymers and sealing the energized polymers in storage.

FIG. 10 is a perspective view of a production line with a rotating ultrasound apparatus that can energize moving polymers from the radial side of an ultrasound tip.

FIG. 11 is a cross-sectional view of a production line with a rotating ultrasound tip capable of energizing moving polymers from the radial side of the ultrasound tip.

FIG. 12 is a cross-sectional view of a production line with an ultrasound tip in a fixed position that can energize moving polymers.

FIG. 13 is a cross-sectional view of a production line with two rotating ultrasound tips capable of energizing moving polymers from the radial side of ultrasound tips.

FIG. 14 is a cross-sectional view of a production line with a rotating ultrasound tip that is capable of energizing moving polymers from the radial side of the ultrasound tip.

### DETAILED DESCRIPTION OF THE INVENTION

The present invention is an apparatus and methods for pain relief using polymers energized by exposure to ultrasonic waves, and said polymers are capable of storing the energy imparted to them from ultrasound exposure. Preferred embodiments of the present invention in the context of an apparatus and methods are illustrated in the figures and described in detail below.

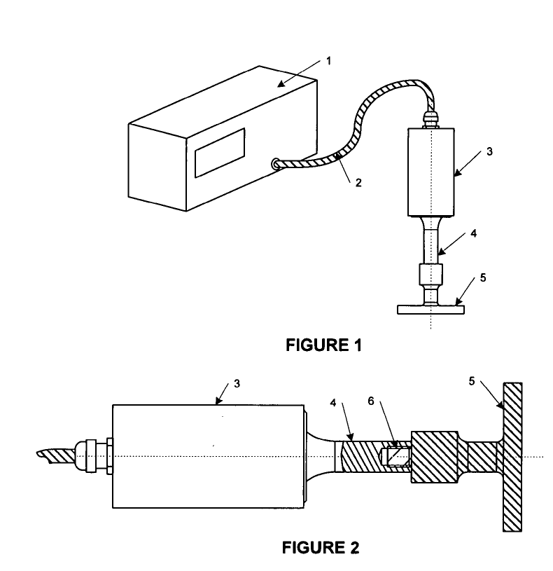

FIG. 1 is a perspective view for an ultrasound apparatus capable of energizing polymers according to the present invention. The ultrasound apparatus comprise an ultrasound power generator 1, a power supply cord 2, an ultrasonic transducer 3, an ultrasound horn 4, and an ultrasound tip 5.

FIG. 2 is a cross-sectional view of the ultrasound transducer 3 with accompanying ultrasound horn 4 and ultrasound tip 5 that is depicted in FIG. 1. The ultrasonic transducer 3 is connected to the ultrasound horn 4. The ultrasound horn 4 is mechanically connected to an ultrasound tip 5 by threading or other means 6. The preferred embodiment comprises an ultrasound tip 5 that is directly connected to the ultrasound horn 4 by a mechanical interface;

alternative embodiments could have the ultrasound tip 5 directly connected to the ultrasound horn 4 to comprise a single piece without a mechanical interface.

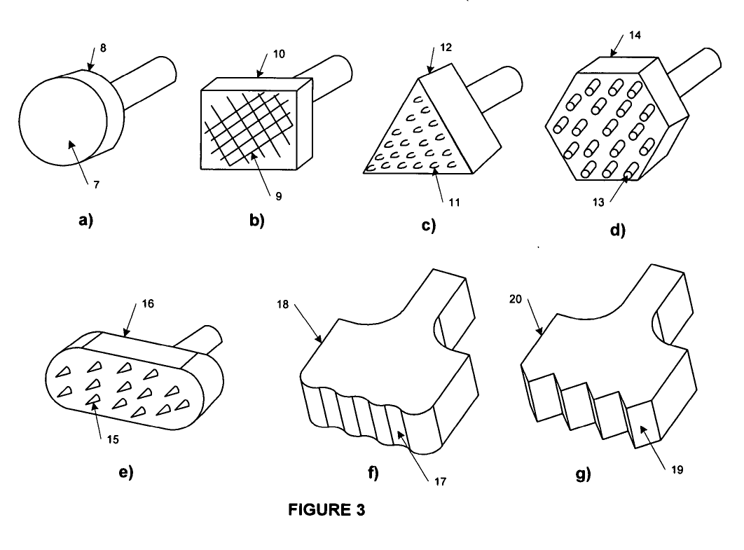

FIGS. 3a-3g are front-views of ultrasound tips that can be used with the ultrasound apparatus depicted in FIG. 1. FIG. 3a is an ultrasound tip that has a smooth front surface 7 and a circular peripheral boundary 8. FIG. 3b is an ultrasound tip that has a knurled front surface 9 and a rectangular peripheral boundary 10. FIG. 3c is an ultrasound tip that has a pyramidal front surface 11 and a triangular peripheral boundary 12. FIG. 3d is an ultrasound tip that has a cylindrical front surface 13 and a polygonal peripheral boundary 14. FIG. 3e is an ultrasound tip that has a spiky front surface 15 and an elliptical peripheral boundary 16. FIG. 3f is an ultrasound tip that has a waved front surface 17 and a rectangular peripheral boundary 18. FIG. 3g is an ultrasound tip that has a grooved front surface 19 and a rectangular peripheral boundary 20. These are only examples of front surfaces and peripheral boundaries of ultrasound tips that can be used with the ultrasound apparatus according to the present invention. Other front surfaces and peripheral boundaries may be similarly effective. Furthermore, any front surface can be mixed and matched with any peripheral boundary.

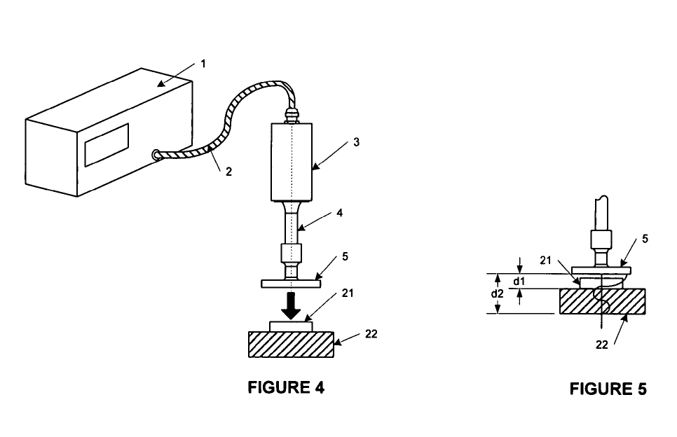

FIG. 4 is a perspective view of an ultrasound apparatus capable of energizing polymers through direct contact with a polymer. The ultrasound apparatus comprises an ultrasound power generator 1, a power supply cord 2, an ultrasonic transducer 3, an ultrasound horn 4, and an ultrasound tip 5. The ultrasound tip 5 delivers ultrasonic energy to the polymer 21 that is located on base material 22. Examples of polymer 21 to use include, but are not limited to, crystalline polymers, amorphous polymers, polymer alloys, or any other polymers currently approved for use in medical devices or food contact substances by the Federal Food and Drug Administration. Other polymers not currently approved may be similarly effective. The recommended polymer to use is a crystalline polymer. Examples of base material 22 on which to place the polymer 21 during delivery of ultrasonic waves include, but are not limited to, metals, polymers, elastomers, ceramics, rubbers, fabrics, composite materials, or any other similarly effective base materials or a combination thereof An energized polymer 21 can be placed on a user to provide an analgesic effect.

FIG. 5 is a detailed view of the ultrasound apparatus depicted in FIG. 4 that can energize polymers through direct contact with a polymer. The ultrasound tip 5 delivers ultrasonic waves to the polymer 21 that is located on base material 22. Depending on the base material 22 used, ultrasound waves can travel through the base material 22 as shown in the sine wave that illustrates the emanated ultrasound energy. Depending upon the ultrasound parameters, reflection can occur both at the upper and lower surfaces levels of the base material 22; reflection of the ultrasonic waves can also occur at the lower surface level of the polymer 21.

Finally, the amount of reflection may vary depending on the distance dl between the ultrasound tip 5 and the lower surface level of polymer 21 and may also vary depending on the distance d2 between the ultrasound tip 5 and the lower surface level of the base material 22. Reflection of ultrasonic waves can result in a polymer being double exposed to ultrasonic waves capable of energizing the polymer.

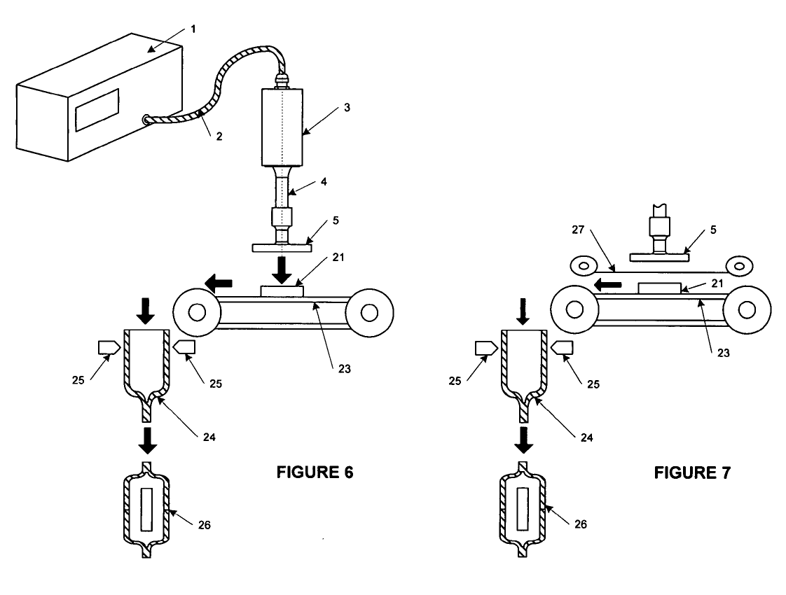

FIG. 6 is a perspective schematic view of a production line with an ultrasound apparatus capable of energizing polymers through direct contact. The ultrasound apparatus comprises an ultrasound power generator 1, a power supply cord 2, an ultrasound transducer 3, an ultrasound horn 4, and an ultrasound tip 5. The ultrasound tip 5 delivers ultrasonic waves to the polymer 21 that is located on base material 23. After being energized by exposure to ultrasonic waves, the polymer 21 moves down the production line and into storage material 24 that is secured by sealers 25, resulting in a sealed packet 26. Examples of storage material 24 to use include, but are not limited to, plastic bags, plastic sleeves, film, or fabric. Other storage materials may be similarly effective. The energized polymer 21 can be placed on a user to provide an analgesic effect. The use of the storage material 24 allows the polymer 21 to store energy, thus allowing the polymer 21 to be removed from the sealed packet 26 at a future time to be placed on a user provide an analgesic effect.

FIG. 7 is a perspective schematic view of a production line with an ultrasound apparatus capable of energizing polymers through a coupling medium. The ultrasound tip 5 delivers ultrasonic energy though a coupling medium 27 to the polymer 21 that is located on base material 23. Examples of coupling medium 27 include, but are not limited to, film, liquid, gel, or ointment. Other coupling mediums can be similarly effective. After being energized by exposure to ultrasonic waves, the polymer 21 moves down the production line and into storage material 24 that is secured by sealers 25, resulting in a sealed packet 26. Examples of storage material 24 to use include, but are not limited to, plastic bags, plastic sleeves, film, or fabric. Other storage materials may be similarly effective. The energized polymer 21 can be placed on a user to provide an analgesic effect. The use of the storage material 24 allows the polymer 21 to store energy, thus allowing the polymer 21 to be removed from the sealed packet 26 at a future time to be placed on a user provide an analgesic effect.

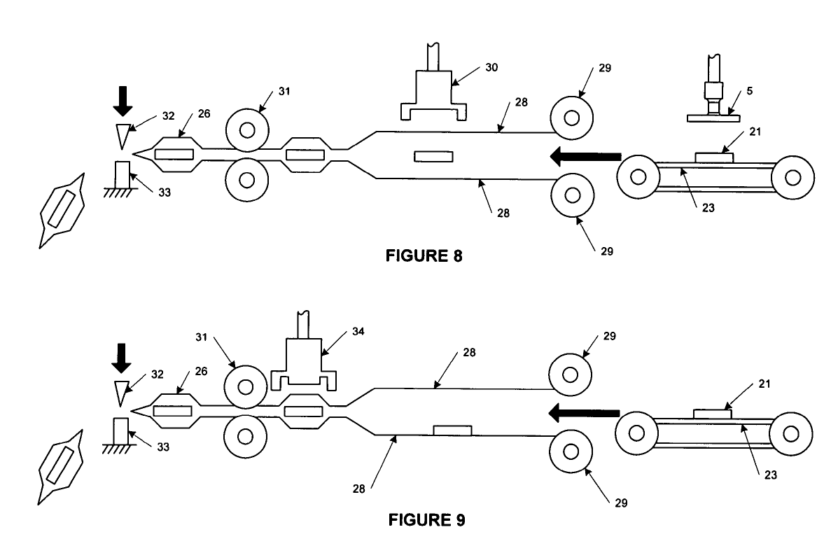

FIG. 8 is a perspective schematic view of a production line with an ultrasound apparatus capable of energizing polymers and with a separate device to seal polymers in storage. The ultrasound horn 5 delivers ultrasonic waves to the polymer 21 that is located on base material 23. After being energized by exposure to ultrasonic waves, the polymer 21 moves down the production line and into storage material 28 that is released from storage material spools 29. The storage material 28 may consist of one adhesive and one non-adhesive side, or it may also consist of two non-adhesive sides. Examples of storage material 28 to use include, but are not limited to, plastic bags, plastic sleeves, film, or fabric. Other storage materials may be similarly effective. The polymer 21 is then sealed in the storage material 28 by ultrasonic welding with ultrasound waves delivered from ultrasound tip 30. Ultrasound welding is an example of a sealing method; other methods, such as heat, may be similarly effective. The sealed packet 26 moves down the production line by driving wheels 31 where it is cut into an individual section by blade 32 contacting cutting block 33. Other methods and devices may be similarly effective in separating the sealed packet 26. The energized polymer 21 can be placed on a user to provide an analgesic effect. The use of the storage material 28 allows the polymer 21 to store energy, thus allowing the polymer 21 to be removed from the sealed packet 26 at a future time to be placed on a user to provide an analgesic effect.

FIG. 9 is a perspective view of a production line with an ultrasound apparatus capable of both energizing polymers and sealing the energized polymers in storage. Polymer 21 moves down production line into storage material 28 that is released from storage material spools 29. The ultrasound tip 34 then serves a dual function: the tip 34 delivers ultrasonic waves to the polymer 21 that is in the storage material 28, and the tip 34 also delivers ultrasonic waves to the storage material 28 in order to seal the polymer 21 in the storage material 28. Energizing the polymer 21 can occur before, during, or after ultrasound energy is delivered to seal the polymer 21 in the storage material 28. The sealed packet 26 moves down the production line by driving wheels 31 and then is cut into an individual section by blade 32 contacting cutting block 33. Other methods and devices may be similarly effective in separating the sealed packet 26. The energized polymer 21 can be placed on a user to provide an analgesic effect. The use of the storage material 28 allows the polymer 21 to store energy, thus allowing the polymer 21 to be removed from the sealed packet 26 at a future time to be placed on a user to provide an analgesic effect.

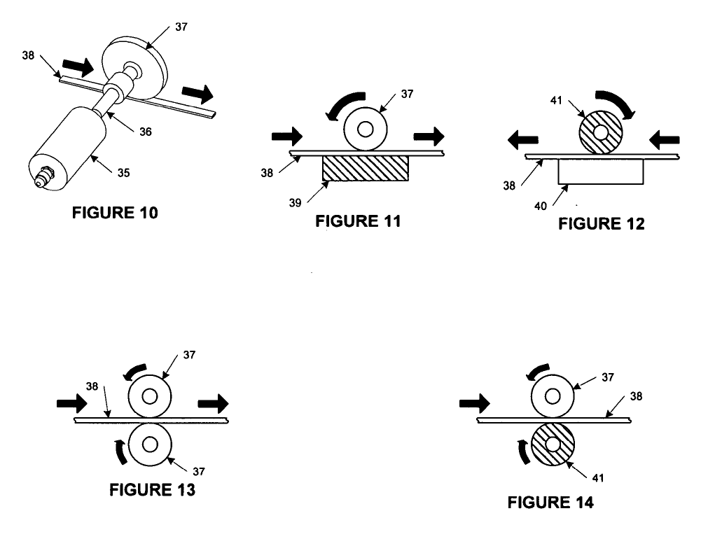

FIG. 10 is a perspective view of a production line with a rotating ultrasound apparatus that can energize moving polymers from the radial side of an ultrasound tip. The ultrasound apparatus consists of an ultrasonic transducer 35 that is connected to the ultrasound horn 36, and the ultrasound horn 36 is connected to the ultrasound tip 37. The ultrasound apparatus rotates and energizes the polymer 38 from the radial side of the ultrasound tip 37 as the polymer 38 moves down the production line. The recommended peripheral boundary for an ultrasound tip 37 on a rotating ultrasound apparatus is circular. Other peripheral boundaries may be similarly effective. The recommended radial surface for the ultrasound tip 37 is smooth. Other radial surfaces such as knurled, waved, or grooved (not shown) can be similarly effective. This production line method allows for large sections of polymer to be sonicated at once because after the moving polymer 38 has been energized, it can be cut into individual sections and sealed for use at a future time.

FIG. 11 is a cross-sectional view of a production line with a rotating ultrasound tip capable of energizing moving polymers from the radial side of the ultrasound tip. The moving polymer 38 moves down the production line to be energized by ultrasonic waves delivered from the radial side of the rotating ultrasound tip 37. There is base material 39 that is located in a fixed position on the other side of the moving polymer 38 from the rotating ultrasound tip 37.

Once the moving polymer 38 has been energized, it can be cut into individual sections and sealed for use at a future time.

FIG. 12 is a cross-sectional view of a production line with an ultrasound tip in a fixed position that can energize moving polymers. The moving polymer 38 moves down production line to be energized by ultrasonic waves delivered from the radial side or distal end of the ultrasound tip 40 that is located in a fixed position. There is base material 41 located on the other side of the moving polymer 38 from the fixed ultrasound tip 40. The base material 41 rotates as the polymer 38 moves down the production line. Once the moving polymer 38 has been energized, it can be cut into individual sections and sealed for use at a future time.

FIG. 13 is a cross-sectional view of a production line with two rotating ultrasound tips capable of energizing moving polymers from the radial side of ultrasound tips. The moving polymer 38 moves down production line to be energized on each side by ultrasonic waves delivered from the radial sides of the rotating ultrasound tips 37. There no is base material in this production line. Once the moving polymer 38 has been energized, it can be cut into individual sections and sealed for use at a future time.

FIG. 14 is a cross-sectional view of a production line with a rotating ultrasound tip that is capable of energizing moving polymers from the radial side of the ultrasound tip. The moving polymer 38 moves down the production line to be energized by ultrasonic waves delivered from the radial side of the rotating ultrasound tip 37. There is base material 41 located on the other side of the moving polymer 38 from the rotating ultrasound tip 37. The base material 41 also rotates as the polymer 38 moves down the production line. Once the moving polymer 38 has been energized, it can be cut into individual sections and sealed for use at a future time.

The frequency range for the ultrasonic waves capable of energizing a polymer is approximately 15 kHz to approximately 40 MHz, with a preferred frequency range of approximately 20 kHz-approximately 40 kHz. The recommended low-frequency ultrasound value is approximately 30 kHz and the recommended high-frequency ultrasound value is approximately 3 MHz. The amplitude of the ultrasound waves can be 1 micron and above. The preferred amplitude range for low-frequency ultrasound is approximately 50 microns to approximately 60 microns, and the recommended amplitude value for low-frequency ultrasound is approximately 50 microns. The preferred amplitude range for high-frequency ultrasound is approximately 3 microns to approximately 10 microns, and the recommended amplitude value for high-frequency ultrasound is approximately 3 microns. The time of sonication will vary based on factors such as the ultrasound frequency, amplitude, intensity, the type of polymer, the thickness of polymer, the type of base material, the thickness of base material, etc.

Ultrasonic waves are delivered from an ultrasound apparatus to a polymer to energize the polymer. Ultrasonic waves can be delivered by either direct contact, through a coupling medium, or without contact. Ultrasonic waves can also be delivered from either the distal end or the radial side of the ultrasound horn/tip. The shape of the ultrasound tip used may vary. The peripheral boundary may be circular, rectangular, triangular, polygonal, elliptical, or another similar shape or combination of shapes. The front surface of the ultrasound tip may be smooth, knurled, pyramidal, cylindrical, spiky, waved, grooved or another similar surface or combination of surfaces. The preferred shape of the ultrasound tip is a smooth front surface with a rectangular peripheral boundary, but other shapes can also be similarly effective.

The polymer may be placed on surface material while being energized by exposure to ultrasonic waves. The surface materials that may be used vary from metals, polymers, elastomers, ceramics, rubbers, fabrics, composite materials, or any other similarly effective surface materials or a combination thereof. The size and thickness of the surface material can also vary. Besides acting as a base while the polymer is being energized, the surface material can also serve an additional purpose. Depending upon the surface material used and the parameters of the ultrasound waves delivered, ultrasound waves can reflect off of the surface material and back onto the polymer once again, thus resulting in the polymer being double exposed to ultrasonic waves capable of energizing the polymer. The ultrasonic waves can also reflect off the lower surface level of the polymer itself. The polymer can also be energized by means other than ultrasound such as UV, microwave, laser, electricity, RF, sun, light, magnetic/electromagnetic, etc.

The polymer may be placed in storage material before, after, or while being energized by ultrasonic waves. The polymer can be energized and then dropped into storage material, fed into storage material, or any other method to store an energized polymer. The polymer can also be fed into storage material so that it can energized and sealed simultaneously. Finally, the polymer can be sealed in its storage material and then it can be energized through the storage material.

The energized polymer can be placed on a user to provide an analgesic effect. The energized polymer can be removed from the storage material at a future to be placed on a user to provide an analgesic effect. The recommended use of the energized polymer is to place the energized polymer directly on the user's skin, and preferably to place the energized polymer on the user's pain area.

Although specific embodiments and methods of use have been illustrated and described herein, it will be appreciated by those of ordinary skill in the art that any arrangement that is calculated to achieve the same purpose may be substituted for the specific embodiments and methods shown. It is to be understood that the above description is intended to be illustrative and not restrictive. Combinations of the above embodiments and other embodiments as well as combinations of the above methods of use and other methods of use will be apparent to those having skill in the art upon review of the present disclosure. The scope of the present invention should be determined with reference to the appended claims, along with the full scope of equivalents to which such claims are entitled.

1. A method for creating ultrasound energized polymers, comprising the steps of:
placing a single polymer article on a base surface, apart from other polymer articles;
generating ultrasonic waves with an ultrasound apparatus having an ultrasound tip, the ultrasonic waves having an intensity configured for energizing a polymer;
delivering the ultrasonic waves to the single polymer article by directly contacting and pressing the single polymer article against the base surface with the ultrasound tip to create an energized polymer article, the polymer article being selected from a group approved for use in a medical device by the Food and Drug Administration; and
packaging the energized polymer article in a sealed packet for purposes of storage and later use.

2. (canceled)

3. The method according to claim 1, wherein the ultrasound frequency is in the range of approximately 15 kHz-approximately 40 MHz.

4.-7. (canceled)

8. The method according to claim 1, wherein the ultrasound amplitude is at least 1 micron.

9.-15. (canceled)

16. The method according to claim 1, wherein the ultrasound waves are delivered to the polymer for at least of 0.1 seconds.

17.-18. (canceled)

19. The method according to claim 1, wherein the base material includes one of metal, polymer, elastomer, ceramic, rubber, fabric, composite material, and combinations thereof.

20. (canceled)

21. The method according to claim 1, wherein the sealed packet is a plastic bag.

22.-27. (canceled)

28. The method according to claim 1, wherein polymers move down a production line system to be directly energized by an ultrasound apparatus/system and then placed in a means of storage.

29. (canceled)

30. The method according to claim 1, wherein polymers move down a production line to be energized by an ultrasound apparatus/system and then sealed in a means of storage by a separate apparatus/system.

31. The method according to claim 1, wherein polymers move down a production line to be energized and sealed in a means of storage by the same apparatus/system.

32.-98. (canceled)

99. A method for creating ultrasound energized polymers, comprising the steps of:
placing a single polymer article having a thickness on a base having a thickness, the placing being performed apart from other polymer articles;
generating ultrasonic waves with an ultrasound apparatus having an ultrasound tip, the ultrasonic waves having an intensity configured for energizing a polymer;
delivering the ultrasonic waves to the single polymer article by directly contacting and pressing the single polymer article against the base with the ultrasound tip to create an energized polymer article; and
packaging the energized polymer article in a sealed packet for purposes of storage and later use,
wherein, ultrasound parameters, the polymer thickness, and the base thickness are selected to doubly expose the polymer to ultrasound waves.
